(19) United States
(12) Patent Application Publication
(10) Pub. No.: US 2006/0276768 A1
(43) Pub. Date: Dec. 7, 2006

(54) Container for transporting blood and blood products

(76) Inventors: Randal H. Miller, Peachtree City, GA (US); James S. LeClair, Covington, GA (US); Kenneth F. Wagner, Islamorada, FL (US); Clifford M. Glade, Islamorada, FL (US)

Correspondence Address: LAW OFFICES OF CAROL N. GREEN, P.A.;SUITE 2750, 200 S. BISCAYNE BLVD, MIAMI, FL, 33131, US

(21) Appl. No.: 11/281,548

(22) Filed: Nov. 17, 2005

Related U.S. Application Data
(60) Provisional application No. 60/681,769, filed on May 17, 2005.

(51) Int. Cl. A61B 19/00

(52) U.S. Cl. 604/403

(57) ABSTRACT

The invention is directed to a reusable container for transporting blood and blood products providing the ability to not only transport, but also to monitor and maintain refrigerated blood or blood products at optimal temperature range for more than twenty-four (24) hours without the use of wet or dry ice, or gel packs.

### PRIORITY CLAIM

The present application is a regular Non-Provisional Patent Application claiming priority to U.S. Provisional Patent Application, Container for Transporting Blood and Blood Products, Ser. No. 60/681,769 filed May 17, 2005 and the entire disclosure of the Provisional Application Ser. No. 60/681,769 is incorporated herein by reference.

### FIELD OF THE INVENTION

The invention relates to an improved container for transporting blood and blood products at the required temperature range for extended periods of time without the use of either wet or dry ice, or gel packs, in a durable, reusable container that is capable of maintaining and monitoring the temperature in transit for compliance with certain protocols.

### BACKGROUND OF THE INVENTION

Transporting blood and blood products in a timely manner has always been a problem. Generally, the health care industry has relied on products developed and manufactured for the recreational industry for keeping food and beverages cold as a working solution. However, the recreational industry did not, and could not address the need for documented temperature monitoring for blood products for transfusions that the Food and Drug Administration (FDA) now requires as of 2004. Thus, the healthcare industry is in desperate need for a better solution to transport blood, blood products, human tissues, stem cells, and donor organs, which require documented temperature tracking for FDA compliance.

Spoilage and the risk of infection from stale blood, blood products, tissues, cells or donor organs are also of paramount concern. The FDA requires that blood and blood products being transported must be maintained at a certain temperature range. For instance, packed red blood cells should be maintained at a temperature range of 1° C. to 6° C.; fresh plasma thawed (for use) requires a temperature range of 1° C. to 6° C.; platelet concentrates must be maintained at 20° C. to 24° C.; and fresh frozen plasma must be stored at or below −25° C.

Additionally, transporting blood and blood products require that certain protocols be met in order to prevent exposure, damage or contamination while in transit. Many blood banks try to solve this problem by transporting blood and blood products in a leak proof primary container placed in a secondary container, such that the specimen is protected from inadvertent puncture or contamination. The units of red blood cells are then packed on wet or dry ice or a chemical coolant to be transported in Styrofoam® containers.

However, one of the limitations of this method of transportation is that wet ice is messy, often spills during transportation, and re-icing while in transit is sometimes challenging in remote areas. Dry ice offers its own complications as it can be hazardous where if for example loose dry ice inadvertently touches the blood or blood product, it may freeze the blood or blood product at the area of contact and damage the entire unit. Additionally, dry ice is sometime unavailable in certain parts of the world. These factors only serve to further limit the ability to transport blood and blood products for prolonged periods. To alleviate this problem, frozen gel packs have been used to help maintain required temperatures. However, gel packs are susceptible to leaks and are therefore unreliable.

Another disadvantage is that the blood and blood products have limited viability over prolonged periods even if transported at the required temperature range. Red blood cells when stored at the required temperature range are viable for up to six (6) hours, while blood platelets are limited to four hours expiration time. Conventional insulated shipping containers do not address this problem, which can be further complicated when blood or blood products are being transported internationally. Thus, there still exists a need to be able to transport blood and blood products efficiently and safely for periods lasting longer than four to six hours without the use of either wet or dry ice, or gel packs in a durable, reusable container that is capable of maintaining or monitoring the temperature in transit for compliance with certain protocols.

### SUMMARY OF THE INVENTION

The invention is directed to a reusable container for transporting blood and blood products providing the ability to not only transport, but also to monitor and maintain the refrigerated blood or blood products at the necessary temperature range for more than twenty-four (24) hours without the use of wet or dry ice, or gel packs. The container is capable of transporting 1 to 2, 1 to 4, or 1-6 or more units of blood from the blood bank to Operating Room, Emergency Room, dialysis, cancer center, home health care facilities or any other facility where blood is used.

The container comprises of a waterproof outer case, which may be constructed from hard, durable, rustproof material or a soft, flexible, lightweight material that is durable and stain resistant. The outer case provides for varied means for lifting and towing the container, which may be singularly employed or in combination thereof, for example a reinforced handle on the top of the container, wheels, a telescopic handle, non-retractable handles and shoulder straps.

The exterior of the container contains at least one information sleeve adapted to hold identification information concerning the blood or blood product being transported. A patient's name, social security or identification number, surgeon, blood type, date the blood or blood product left the blood bank, scheduled date of delivery, number of units of blood, delivery operating room number, surgeon, doctor and medical procedure can be documented and stored in the information sleeve.

The container further comprises of an insulated layer having four side walls, a bottom portion, and an open top defining a receptacle area for receiving the insulation deck. The walls and the bottom portion of the insulated layer have a predetermined thickness to thermally insulate the container. The insulated layer substantially surrounds the inner perimeter of the outer case. The container also includes an insulation deck that provides for a bottom portion being connected to walls substantially surrounding the insulation layer forming a seamless well for spill proofing and for receiving the cooling elements. The insulation deck is secured to the outer case by appropriate securing means forming a receptacle area for receiving the cooling elements.

The cooling elements are an important feature of the container in accordance with the present invention. The cooling elements comprise of four modular units shaped and sized to securely fit the top, bottom and sides of the inner perimeter of the insulation deck and is adapted to form a receptacle area for housing an inner caddy. The cooling elements are responsible for keeping the inner caddy at the required temperature (1° C. to 6° C.), and is made from strong, durable and lightweight material. A refrigerant material is disposed in all or part of the cooling elements and may include a phase change gel. The cooling elements are adapted to, when frozen and conditioned, maintain the required temperature for extended periods of time, i.e. 24+ hours. The cooling elements must be frozen prior to each day's use in any standard or commercial freezer for up to 24 hours prior to each use. The cooling elements are stackable and provides for easy storage in a freezer. After removal from the freezer, the cooling elements must be conditioned prior to use and insertion into the container. The object of conditioning the cooling elements is to allow it to transition from the temperature of the freezer to a temperature proximal to the desired phase change temperature. This is achieved simply by exposing the cooling elements to the ambient room temperature by placing it out on a counter (not in the insulated case), for approximately 30 to 60 minutes.

The container includes an inner caddy, which provides for a bottom portion connected to sidewalls, a front wall, a back wall and a middle divider. Lifting means are provided from lifting and removing the inner caddy from the container. Lifting means may include a handle, strap, tab, or anything that can be grasped or is generally known and used by those skilled in the arts. In one embodiment, the walls include one or more top portions that are hingedly connected to a lid extending outwardly open from the receptacle area to form gull wings. In another embodiment, the inner caddy opens from front to back providing access to its contents. The inner caddy is removable with cleaning vents, allowing for self-drainage and ease of cleaning. The inner caddy is conveniently sized such that it may be stored in a standard or commercial refrigerator until the blood or blood product is ready for use.

The container is further equipped with a receiving area for a temperature data-logging device which comprises of at least one probe for monitoring, collecting and reporting data concerning the blood or blood units being transported. Suitable temperature data-logging devices may include for example a Validator, which consists of proprietary technology currently available through American Thermal Wizard International, Inc. The temperature data-logging device is programmable to meet the compliance requirements and will continuously monitor the temperature of the blood or blood products while it is housed in the container. The information once logged can be graphed or logged and is capable of being downloaded into Microsoft Word® or Excel®. The temperature data-logging device can be programmed to either transmit an alarm or a page if the temperature range falls or exceeds the set parameters.

For a further and more fully detailed understanding of the present invention, various objects and advantages thereof, reference is made to the following detailed description and the accompanying drawings.

### BRIEF DESCRIPTION OF THE DRAWINGS

Further objectives and advantages of the invention will become more apparent from the following description and claims and from the accompanying drawings herein.

FIG. 1 depicts a container for transporting blood and blood products.

FIG. 2 depicts an external view of one embodiment of the container for transporting blood and blood products.

FIG. 3 depicts an alternate embodiment of the container for transporting blood and blood products.

FIG. 4 depicts an embodiment of the cooling elements of the invention.

FIG. 5 depicts one embodiment of the inner caddy.

### DESCRIPTION OF PREFERRED EMBODIMENTS

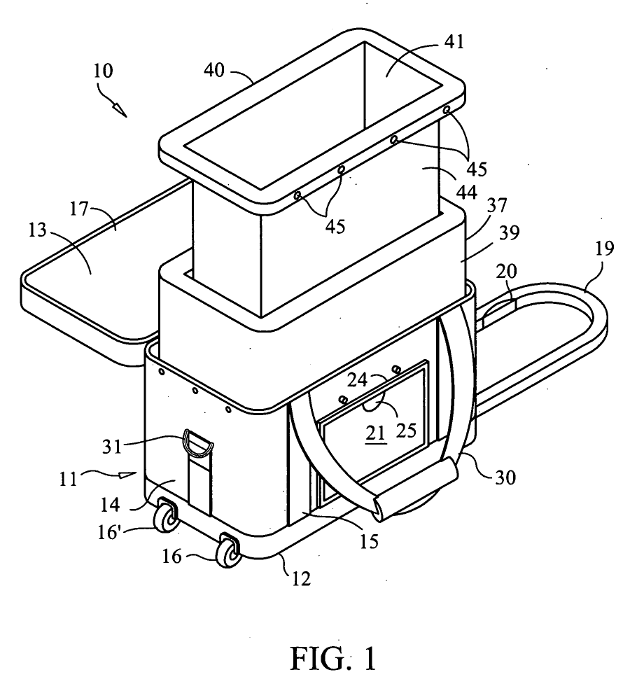

By reference to the exemplary drawings in detail wherein like numerals indicate like elements throughout the various views, there is shown in FIG. 1 the container 10 for transporting blood and blood products in accordance with the invention.

The container 10 comprises of an outer case 11 that may be formed from soft, flexible, lightweight material like canvas, or hard rigid material for more durability, or any other suitable materials generally known and used by persons skilled in the art. In both embodiments, the outer case 11 is made of waterproof, stain resistant, rustproof material, is easily cleaned, and reusable. The container 10 may be made in different sizes depending on the number of blood or blood units that are being transported. In one embodiment, the container 10 holds 1-2 units of blood or blood products. In another embodiment, the container 10 holds 1-4 units of blood. Alternatively, the container 10 holds either 1-6, or more units of blood or blood products.

The outer case 11 comprises of a base 12 which may be formed from a reinforced or stiff material, or of other suitable materials generally known and used by persons skilled in the art. In an alternate embodiment, the base 12 may include a rigid base insert covered with a flexible material. The outer case 11 further comprises of ends 14 and 14′ (not shown) that are connected to the base 12, with sides 15 and 15′ (not shown) extending between the ends 14 and 14′ (not shown).

The container 10 provides for a lid 17, which is hingedly attached to the sides 15 and 15′ (not shown) of the outer case 11 and is closeable by twist latches 30, 30′. Preferably, at least one twist latch 30 is lockable. Alternatively, other suitable forms of closeable means such as hooks, loops, snap fasteners, buttons, Velcro or zippers may be employed to close the lid 17.

FIG. 3 depicts one embodiment of the invention wherein the lid 17 may be removable from the container 10 and is hingedly attached to one of the sides 15, and may be closable with at least one zipper 29 and 29′. Alternatively, the zipper 29 may extend around the entire periphery of the lid 17 such that the lid 17 is completely removable. In another embodiment of the invention, the lid 17 may have a zipper 29 extending around two ends 14, 14′ and one (1) side 15 so that the lid 17 may be hinged at its attached side 15 providing maximum opening without being completely removable. However, it will be appreciated by those skilled in the arts of the invention that any form of a lid 17 may be employed.

In one embodiment, the lid's interior 13 comprises of closed cell PE foam insert that acts as a water barrier to protect the interior contents of the container 10. In another embodiment, insulation is added to the lid's interior 13 to prevent the transmittal of external heat thus protecting the interior contents of the container 10. In an alternate embodiment, a cooling module is added to the lid's interior 13 wherein refrigerant material is disposed within further enhancing the performance of the container 11.

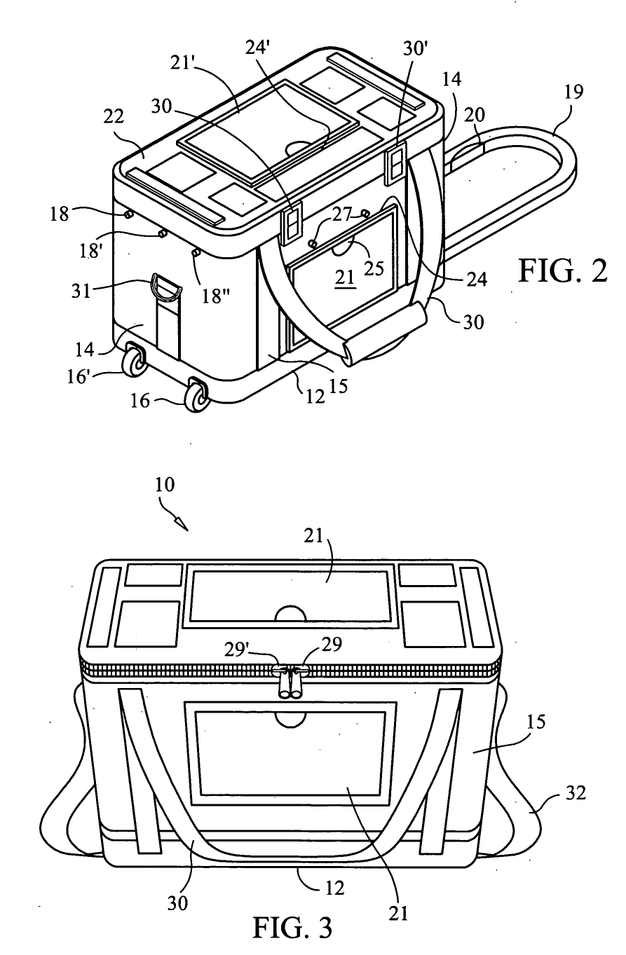

As shown in FIG. 2, a wheel assembly 16, 16′ and anchor screws 18, 18′, 18″ located on one of the opposing ends 14 provides stabilizing means for the container 10 when stood on its end 14. The stabilizing means allow the container 10 to be placed flat on its base 12 without the container 10 tipping over. Other stabilizing means may include at least a foot, claw or any other stabilizing means that is generally known and used by persons skilled in the art. In one embodiment of the invention, a telescopic handle 19 may be provided adjacent to the base 12 for pulling the container 10 on the wheel assembly 16, 16′. The telescopic handle 19 is capable of gradually extending to the desired height from the top end 14′ of the container 10 when a push button 20 is pressed. The push button 20 also serves to retract the telescopic handle 19 when pressed.

The container 10 also preferably comprises of at least one pair of non-retractable handles 30 and 30′ (not shown) providing alternate means for picking up and carrying the container 10. As shown in FIGS. 1 & 2, the outer case 11 provides rings 31 and 31′ (not shown) for a shoulder strap 32 (not shown) to be attached for alternate means for picking up and carrying the container 10. FIG. 3 shows one embodiment of the invention where the shoulder straps are attached to the outer case 11. In certain embodiments of the invention, an additional reinforced handle 33 (not shown) is attached to the top of the lid's exterior 22 providing yet another means for picking up and carrying the container 10.

As shown in FIG. 1, the outer case 11 may include at least one identification sleeve 21 which is adapted to secure and hold documentation concerning the blood or blood products being transported, which may include a patient's name, social security or identification number, physician, blood type, date the blood or blood product left the blood bank, scheduled date of delivery, number of units of blood being transported, delivery destination, etc. The identification sleeve 21′ is made of a clear or transparent plastic with an opening 24 adapted with a finger notch 25 for easy insertion and removal of documentation.

As shown in FIG. 2, in one embodiment of the invention the outer case 11 has more than one identification sleeve 21, 21′, one on the side 15 of the outer case 11, and the other on the lid's exterior 22. It is understood by those practicing the arts that there can be as many identification sleeves 21, 21′ as desired. As shown in FIG. 2, the identification sleeve 21 may be secured to the side of the container 10 by anchor screws 27, 27′, 27″, 27′″, or any other securing means that are generally known and used by those skilled in the pertinent arts, including but not limited to, screws, nails, bolts, paste, glue and Velcro. The lid's exterior 11 is adapted for provision of any desired logo or other identification marks or notations.

FIG. 1 depicts an outer case 11 adapted to form a receptacle area for an insulated layer 37 made of suitable insulation materials to shield the interior contents of the container 10 from external temperature. Suitable insulation materials may include a substantially rigid insulating material having a relatively low thermal conductivity and being relatively light weight, for example Minicel®, Volara®, Neoprene, Polystyrene (Styrene), Polyethylene, VIP (Vacuum Insulation Panel), ABS and Coroplast®, TempShield™ and SPACE AGE®. Preferably, the insulated layer 37 is formed from rigid polyurethane.

The insulated layer 37 includes a bottom portion 38 (not shown) connected to walls 39 substantially surrounding the inner perimeter of the outer case 11. The insulated layer 37 has a predetermined height to substantially engage or abut the top of the container 10 while substantially minimize the air space remaining above the container 10. The outer case 11 also includes an insulation deck 40, which may comprise of a PE plastic insert shaped to form a receptacle area 41 for the cooling elements 46, 46′, 47, 47′ (not shown). The insulation deck 40 substantially surrounds the insulated layer 37 and includes a bottom portion 43 (not shown) being connected to the walls 44 in a seamless well for spill proofing and to receive the cooling elements 46. After the insulated layer 37 is placed in the outer case 11, the insulation deck 40 is inserted in the outer case 11 and substantially engages the walls 39 and bottom portion so as to substantially seal the insulated layer 37 to the outer case 10. In one embodiment of the invention, the insulation deck 40 is secured to the outer case 11 by anchor screws 45. It is understood by those skilled in the arts that other securing means such as nails, glue, nuts and bolts may be applied to secure the insulation deck 40 to the outer case 11.

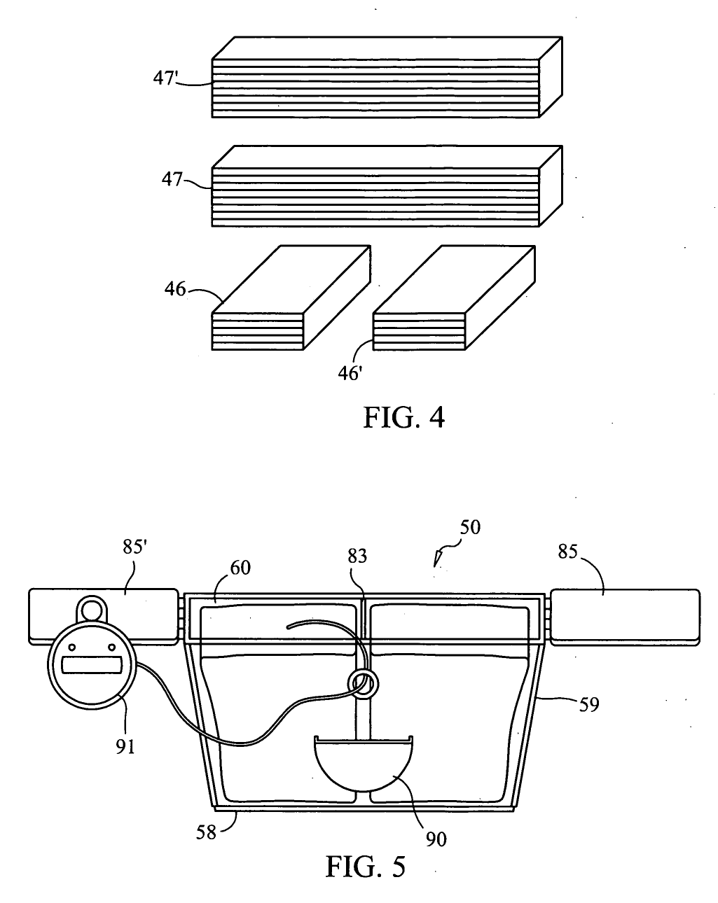

FIG. 4 depicts the cooling elements 46, 46′, 47, 47′ of the present invention, which is responsible for keeping the inner caddy 50 (not shown) at the required temperatures of 1° C. to 6° C. The cooling elements 46, 46′, 47, 47′ are made from strong, durable and lightweight material and comprises of at least four modular units with refrigerant material disposed within each modular unit. The cooling elements 46, 46′, 47, 47′ further comprise of two sides 46 and 46′ and a top 47 and bottom 47′. The two sides 46 and 46′ are sized and shaped to securely fit the interior sides of the insulation deck 40, while the top 47 and bottom 47′ are sized and shaped to fit the bottom and top of the insulation deck 40. The cooling elements 46, 46′, 47, 47′ further comprises of a receptacle area 48 (not shown) for receiving the inner caddy 50.

The cooling elements 46, 46′, 47, 47′ must first be properly frozen in a commercial or home freezer at temperatures set at or below −10° C. for a minimum of 24 hours, and then conditioned (allowed to stand at room temperature for 30-60 minutes after removal from the freezer) prior to use. If the cooling elements 46, 46′, 47, 47′ are already partially frozen, twelve (12) hours should prove sufficient. The cooling elements 46, 46′, 47, 47′ are stackable and easily stored in the freezer. The cooling elements 46, 46′, 47, 47′ must be refrozen before each day's use. Once the cooling elements 46, 46′, 47, 47′ are frozen and conditioned they are ready for insertion into the container 10. The bottom 47′ is first placed in the bottom of the insulation deck 40 and the sides 46 and 46′ are inserted along the sidewall (not shown) of the insulation deck 40 forming a receptacle area 48 for the inner caddy 50. After the inner caddy 50 is loaded and properly secured within the receptacle area 48, then the top 47 is placed on top of the inner caddy 50.

FIG. 5 depicts one embodiment of the inner caddy 50 made to hold, transport and monitor the temperature of 1 to 6 units of blood or blood products at the required temperature range. The inner caddy 50 is constructed of clear, durable and lightweight material, such as Lucite or polycarbonate material or other like materials that are generally known and used by those skilled in the arts. However, it is understood that any clear, durable material of like properties that is known and generally used by those skilled in the arts may be employed. The inner caddy 50 is removable with cleaning vents 57 (not shown), allowing for self-drainage and easy cleaning. The inner caddy 50 comprises of a bottom portion 58 connected to walls 59.

Preferably, the inner caddy 50 defines a holding area 60 that can accommodate two or more units of blood or blood products at the required temperature range. As best seen in FIG. 5, holding area 60 is defined by sidewalls 80 (not shown), a front wall 81 (not shown), a back wall 82 (not shown), bottom portion 86 (not shown) and a middle divider 83. To store a unit of blood or blood products in the holding area 60, the unit is inserted in an upright position in between one of the straight side walls 80 and the middle divider 83. The sidewalls 80 and middle divider 83 are sized and shaped to hold and secure a unit of blood or blood products placed between them.

In one embodiment of the invention, the holding area 60 is adapted to hold 1 to 2 units of blood. In another embodiment, the holding area 60 is adapted to hold up to four units of blood. In yet another embodiment, the holding area 60 is adapted to hold six or more units of blood or blood products. The sidewalls 80 are hingedly connected to lids 85, 85′, which either extend outwardly open from the holding area 60 to form gull wings or from front to back (not shown). Each lid 85, 85′ is situated on an opposing side of the inner caddy 50. In one embodiment, lifting means are provided for easy removal of the inner caddy 50 from the container 10. Lifting means may include a handle, strap, tab, or anything that can be grasped or is generally known and used by those skilled in the arts. In one embodiment, lids 85, 85′ include an aperture 87 (not shown) adapted to receive the lifting means.

In one embodiment of the invention, the lids 85, 85′ are adapted to receive tamper resistant security tags or clips. If the tamper resistant security tags are still in place at the destination address for the blood or blood products being transported, then the recipient can be assured that the blood or blood products was not removed from the container 10. The tamper resistant security tags provide an effective means of ensuring compliance with certain protocols.

As shown in FIG. 5, the inner caddy 50 provides for a receiving area 90, which is sufficiently wide and deep enough to hold and secure the temperature data-logging device 91. In the preferred embodiment, the receiving area 90 is secured to the front wall 81 of the inner caddy 50. However, it is appreciated by those skilled in the arts that the receiving area 90 may be located anywhere on or near the inner caddy provided that the temperature data-logging device 91 is proximately located near the blood or blood units being transported for monitoring purposes.

The temperature data-logging device 91 comprises of at least one probe 92 (not shown) for monitoring, collecting and reporting data concerning the blood or blood units being transported. The temperature data-logging device 91 is pre-programmable and can be adapted to monitor the temperature of the blood or blood products being transported for 24+ hours to meet the courier or recipient's protocols. In this manner, compliance and performance of the invention can be documented and verified.

After the cooling elements 46, 46′, 47, 47′ have been frozen, conditioned and placed in the container 10, the inner caddy 50 should be removed from the refrigerator with the units of blood or blood products and inserted into the receptacle area 48 formed by the cooling elements 46, 46′, 47, 47′ for receiving the inner caddy 50. The temperature data-logging device 91 is now ready for monitoring and should be positioned in the receiving area 90 with its probe inserted in the holding area 60 at or near the units being transported. The temperature data-logging device 91 may be activated by swiping it with a start magnet. Thereafter, the temperature data-logging device 91 will continuously monitor the units of blood or blood products for up to 24+ hours. The temperature data-logging device 91 can be programmed to either transmit an alarm or a page if the temperature range is outside the set parameters.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements and compositions described herein or in the features or in the sequence of features of the methods described herein without departing from the spirit and scope of the invention as described in the following claims.

What is claimed is:

1. A container for transporting blood and blood products comprising:
an outer case adapted to form a receptacle area for an insulated layer, the insulated layer comprising of walls and a bottom portion substantially engaging an inner perimeter of the outer case and abutting the container's top;
an insulation deck comprising of a bottom portion being connected to walls forming a seamless well wherein the insulation deck substantially engages the walls and bottom portion of the insulated layer so as to substantially seal the insulated layer to the outer case while providing a receptacle area;
cooling elements comprising of sealed modular units, with refrigerant material disposed within the modular units, that are capable of maintaining temperature within a prescribed temperature range for extended periods of time upon being frozen and conditioned prior to use;
an inner caddy comprising of a bottom portion connected to walls defining a holding area that may accommodate at least one unit of refrigerated blood or blood product where the inner caddy further comprises of lifting means; and
a programmable temperature data-logging device for monitoring, collecting and reporting data concerning the blood and blood products being transported.

2. The container of claim 1 wherein the outer case further comprises of hard, durable material.

3. The container of claim 1 wherein the outer case further comprises of flexible material that is durable.

4. The container of claim 1 wherein the outer case further comprises of means for lifting and towing the container.

5. The container of claim 4, wherein the lifting and towing means further comprises of a reinforced handle.

6. The container of claim 4, wherein the lifting and towing means further comprises of a wheel assembly.

7. The container of claim 4, wherein the lifting and towing means further comprises of a telescopic handle.

8. The container of claim 4, wherein the lifting and towing means further comprises of a non-retractable handle.

9. The container of claim 4, wherein the lifting and towing means further comprises of a shoulder strap.

10. The container of claim 1 wherein the outer case further comprises of at least one information sleeve adapted to hold identification information concerning the blood or blood product being transported.

11. The container of claim 1 wherein the outer case further comprises of a lid that is hingedly attached to the outer case with closeable means for closing the container.

12. The container of claim 11 wherein the closeable means further comprises of twist latches.

13. The container of claim 11 wherein the closeable means further comprises of at least one zipper.

14. The container of claim 1 wherein the insulated layer further comprises of insulating material having a relatively low thermal conductivity.

15. The container of claim 1 wherein the insulated layer further comprises of a relatively lightweight material.

16. The container of claim 15 wherein the insulated layer further comprises of polyurethane material.

17. The container of claim 1 wherein the insulation deck is shaped to form a receptacle area for the cooling element.

18. The container of claim 1 wherein the insulation deck further comprises of PE plastic insert.

19. The container of claim 1 wherein the cooling elements are adapted when frozen and conditioned to maintain the required temperature for extended periods of time.

20. The container of claim 19 wherein the cooling elements further comprise of a phase change gel as the refrigerant material.

21. The container of claim 1 wherein the lifting means of the inner caddy includes a handle.

22. The container of claim 1 wherein the inner caddy further comprises of walls that include one or more top portions that are hingedly connected to a lid extending outwardly open from the receptacle area to form gull wings.

23. The container of claim 1 wherein the inner caddy further comprises of walls that that open from front to back.

24. The container of claim 1 wherein the inner caddy is removable with cleaning vents allowing for self-drainage.
